(19) United States
(12) Patent Application Publication
(10) Pub. No.: US 2013/0196934 A1
(43) Pub. Date: Aug. 1, 2013

(54) COMPOSITION FOR PERINATAL AND NEONATAL STROKE

(71) Applicant: Paul Bradley Addis, Cumberland, WI (US)

(72) Inventor: Paul Bradley Addis, Cumberland, WI (US)

(21) Appl. No.: 13/574,185

(22) PCT Filed: Jan. 21, 2011
(86) PCT No.: PCT/US11/22119

§ 371 Date: Oct. 9, 2012

Related U.S. Application Data
(60) Provisional application No. 61/297,174, filed on Jan. 21, 2010.

(51) Int. Cl. A61K 31/7004; A61K 31/355

(52) U.S. Cl. CPC A61K 31/7004; A61K 31/355
USPC 514/23

(57) ABSTRACT

The present invention relates to a novel, non-toxic, prophylactic maternal nutraceutical composition for prevention, treatment, minimization or amelioration of brain damage from perinatal and/or neonatal stroke.

### RELATED APPLICATION

This application claims priority from U.S. Provisional Application Ser. No. 61/297,174, filed Jan. 21, 2010, which application is herein incorporated by reference.

### FIELD OF THE INVENTION

The present invention relates to a novel, non-toxic, prophylactic maternal nutraceutical composition for prevention, treatment, minimization or amelioration of brain damage from perinatal and/or neonatal stroke.

### BACKGROUND OF THE INVENTION

Stroke refers to the sudden attack caused by the reduction of the flow of blood through the brain. Neo- and perinatal stroke, also called hypoxic-ischemic injury or hypoxic-ischemic encephalopathy HIE (hereafter, “neonatal stroke”) occurs in the brain of an infant, commonly occurring during the third trimester or at, shortly before, during or shortly after birth, resulting in brain damage. Numerous complications of the birthing process, as well as neonatal biological variations (e.g., congenital), may induce such strokes and statistically 0.3% of all births involve such complications. Although numerically low, the occurrence of stroke at this delicate stage of life is, most often, devastating to the baby, the family and all medical personnel involved in the case. The medical costs of children who survive neonatal stroke are staggering. In addition, the litigation costs are also overwhelming (for example, one baby settled for $140 million). In terms of malpractice insurance, the obstetrics-gynecology field of medicine is one of the highest, typically about $200,000 per year per doctor. Therefore, neonatal stroke has a significant, detrimental impact on the overall health care costs.

Additionally, neonatal stroke has a number of deleterious effects on the newborn, varying from mild to catastrophic, depending upon the time and severity of the hypoxia. Brain damage during childbirth, though not a common phenomenon, is frequent enough to result in costs to the medical care system of billions of dollars per year. These costs are comprised of health care costs incurred as a result of the compromised health of the baby, if the child survives. Brain damage during childbirth results in death, or if the child survives, cerebral palsy, learning disabilities, mental retardation, seizure disorders, schizophrenia, hearing and visual disorders, and attention-deficit disorders, including hyperactivity. Deafness and some forms of epilepsy have also been reported in neonatal stroke babies. Financially, brain damage is a staggering medical problem.

### SUMMARY OF THE INVENTION

The present invention relates to a novel, non-toxic, prophylactic maternal nutraceutical composition (e.g., for oral administration) for prevention, treatment, minimization or amelioration of brain damage from perinatal and/or neonatal stroke.

One embodiment provides a method comprising administering to a subject in need thereof an effective amount of ribose to reduce risk of neonatal stroke or reduce risk of sequellae of neonatal stroke, wherein the subject in need thereof is an expecting mother. In one embodiment, the sequellae of neonatal stroke is death, cerebral palsy, learning disabilities, mental retardation, seizure disorders, schizophrenia, hearing (e.g., deafness) and visual disorders, attention-deficit disorders (including, for example, hyperactivity), epilepsy or a combination thereof.

Another embodiment provides a method comprising administering to an expectant mother an amount of ribose to improve the growth, vigor, intelligence, overall health, and/or resistance to disease in the neonate.

One embodiment provides a method comprising administering to a subject in need thereof an effective amount of ribose to reduce oxidative stress by reduction of ROS, wherein the subject in need thereof is an expecting mother or a neonate. In one embodiment, the reduction of oxidative stress yields a reduction in tissue and DNA damage and/or a reduction in risk of cancer.

In one embodiment, the ribose provides protection and/or stabilization of cell membranes of neurons and microglia, protection and/or stabilization of membrane-bound mitochondrial creatine kinase (CK) of neurons and microglia and/or maintenance of fluidity of cell membranes.

Another embodiment provides the administration of one or more antioxidants.

Another embodiment provides for the use of the ribose or the compositions of the invention in medical therapy, wherein the therapy is the treatment and/or prevention of neonatal stroke and/or at least one symptom thereof. One embodiment provides for the use of ribose or the compositions of the invention to prepare a medicament for treating or preventing neonatal stroke and/or at least one symptom thereof.

Other aspects of the invention are described in or are obvious from the following disclosure, and are within the ambit of the invention.

### BRIEF DESCRIPTION OF THE FIGURES

FIG. 1 depicts a graph which shows the total antioxidant protection capacity against peroxyl free radicals for each ingredient, the blend, and a serum control.

FIG. 2 provides a dose response graph.

FIGS. 3a-e depict cellular viability and metabolic function under conditions of oxidative stress.

FIGS. 4a-e depict ROS formation under oxidative stress.

### DETAILED DESCRIPTION OF THE INVENTION

The present invention relates to a novel, non-toxic, prophylactic maternal nutraceutical composition (e.g., for oral administration) for prevention, treatment, minimization or amelioration of brain damage from perinatal and/or neonatal stroke.

### Definitions

The following definitions are used, unless otherwise described:

A “subject” is a vertebrate, preferably a mammal, more preferably a human. Mammals include, but are not limited to, humans, farm animals, sport animals and pets. Included in the terms animals or pets are, but not limited to, dogs, cats, horses, rabbits, mice, rats, sheep, goats, cows and birds.

“Perinatal” is defined as the period occurring “around the time of birth,” for example, from about 22 completed weeks (154 days) of gestation to about 7 completed days after birth. The postnatal period begins immediately after the birth of a child and then extends for about six weeks. “Neonatal” is defined as a newborn which is an infant who is within seconds, minutes, hours, days, or up to a few weeks from birth. In medical contexts, newborn or neonate (from Latin, neonatus, newborn) refers to an infant in the first 28 days of life (less than a month old). The term “newborn” includes premature infants, postmature infants and full term newborns.

As used herein, “treat,” “treating” or “treatment” includes treating, reversing, preventing, ameliorating, or inhibiting an injury or disease-related condition or a symptom of an injury or disease-related condition (e.g., neonatal/perinatal stroke).

An “effective amount” generally means an amount which provides the desired effect. For example, an effective dose is an amount sufficient to effect a beneficial or desired result, including a clinical result. The dose could be administered in one or more administrations and can include any preselected amount of the compounds/compositions described herein. The precise determination of what would be considered an effective dose may be based on factors individual to each subject, including size, age, injury or disease being treated and amount of time since the injury occurred or the disease began or the number of babies the mother is carrying (e.g., twins, triplets etc). Doses can vary depending on the mode of administration, e.g., local or systemic.

“Co-administer” can include simultaneous and/or sequential administration of two or more agents.

The terms “comprises”, “comprising”, and the like can have the meaning ascribed to them in U.S. Patent Law and can mean “includes”, “including” and the like. As used herein, “including” or “includes” or the like means including, without limitation.

### Methods of Using the Compositions of the Invention

The composition of the inventions can be used as prophylactic nutraceutical for treatment, prevention, minimization or amelioration of brain damage and death from perinatal and/or neonatal stroke or symptoms/disease caused thereby (e.g., if the child survives, brain damage during childbirth can result in cerebral palsy, learning disabilities, mental retardation, seizure disorders, schizophrenia, hearing and visual disorders, attention-deficit disorders, including hyperactivity, deafness, neonatal encephalopathy caused by hypoxic-ischemic encephalopathy (HIE) and/or epilepsy).

In both adults and newborns, the brain, like the heart and to a lesser extent skeletal muscle, is a dynamic oxidative energy machine. The neonate brain receives “15% of the total cardiac output and 20% of the body's oxygen supply” (1). As such, the brain is extremely dependent upon oxygen and, therefore, even relatively small decreases in oxygen supply can be catastrophic. However, it is believed that tissue damage is not caused by oxygen loss per se, but the derangement of metabolism that occurs as the result of oxygen loss, namely, the loss of high-energy phosphate compounds, mostly adenosine triphosphate (ATP), and creatine phosphate (CP), lactic acid accumulation, loss of purines, and the release of free radicals (2). Thus, it is disclosed herein the retention of ATP, or its building blocks (purines), and simultaneous inhibition of free radicals (and associated tissue damage caused by them), via judicious use of antioxidants and other natural phytochemicals to treat or prevent neonatal stroke. Such therapy/treatment will also lessen damage caused by lactic acid accumulation, by aiding the re-synthesis of ATP.

Nitric oxide (NO) is the subject of many studies because the vasodilatory properties of NO are helpful in preventing stroke and other ischemic disorders. Sources of NO include nitrate, nitrite, L-arginine and other compounds. Antioxidants help to chemically reduce the various sources of NO to safely release NO to the tissue and therefore, antioxidants will play a role in the ameleoration of neonatal stroke where the therapy includes NO as the agent used. Likewise, ribose, by reducing ROS (discussed herein), helps to preserve the ability of NO generators to release NO in the ischemic tissue.

Neonatal stroke may be defined as oxygen deprivation and/or lack of blood flow through the brain's circulatory system. Therefore, the normal metabolism of the brain is upset and this imbalance ultimately causes neuronal damage. The brain is a highly aerobic organ and its metabolic similarity to heart and skeletal muscle, two other highly energetic tissues, are remarkable. All three tissues have the same metabolic system to conserve ATP in a crisis and share the following characteristics (3):

(1) recycle and utilize large amounts of ADP and ATP;

(2) contain plentiful creatine kinase to stockpile excess ATP;
(ATP+C [symbol] ADP+CP);

(3) contain adenylate kinase (myokinase) to aid in regeneration of ATP, at the expense of ADP
(2ADP [symbol] ATP+AMP);

(4) experience catastrophic losses of purines (AMP breakdown) during hypoxic/ischemic attack. The uncharged purines leak out of the cell and are replenished only by de novo synthesis; this is a very slow process in the brain and has no chance to furnish ATP during an acute anoxic event (stroke);

(5) suffer a cessation of the ATP-dependent sodium, potassium, and calcium and magnesium ion shuttles, causing a problematic situation because of the fact that these four minerals and their transfer in and out of the cell is the basis for nerve transmission; and/or

(6) the upsetting of the balance of these minerals also initiates the proteolytic activity that causes the irreversible loss of cell integrity.

The mitochondria of brain, heart and skeletal muscle also share unusual characteristics:

(1) do not produce ATP as the ultimate high-energy compound;

(2) do produce creatine phosphate as the primary high-energy chemical (as the storage form of ATP);

(3) exhibit a robust oxidative energy metabolism, relative to all other tissues except kidney; and/or

(4) contain a specialized form of CK, mitochondrial CK, an enzyme believed to be bound to the outer surface of the inner mitochondrial membrane (4, 5).

The recent trends in cellular energy metabolism research have identified a close linkage between losses of cellular adenosine triphosphate (ATP) and the production of damaging reactive oxygen species (ROS) and other free radicals.

A common treatment objective for the hypoxic newborn is reperfusion, an attempt to increase oxygen supplies to the brain. This should to be conducted immediately if at all possible and with in a one to two-hour period. However, losses of purines negate much of the expected benefit of reperfusion. The reason for this is complex and first involves the “myokinase” reaction, an attempt to synthesize ATP by combining two of the breakdown molecules, 2ADPs, into ATP as follows: 2ADP→ATP+AMP (3). Unfortunately, the appearance of AMP upsets the normal thermodynamic chemical equilibrium of the cell and the cell re-establishes this equilibrium by further breaking apart AMP into its molecular components, including free purines; these uncharged molecules (purines) are able to leak out of the cell and the cellular re-synthesis of these ATP building blocks takes weeks, too tong to ameliorate a crisis (2). Therefore, even if reperfusion is accomplished, it will have diminishing benefits at lengthening time after birth because there are decreasing levels of building blocks (purines) for the synthesis of ATP.

Several attempts to prevent or minimize brain damage have been studied and few, if any, have shown much success. A “cool-cap” technique, involving hypothermia of the brain has been developed. However, the cool-cap seems to show limited benefit, but results are inconsistent. Furthermore, a limited number of hospitals are equipped with a cool-cap device, necessitating the transfer of some compromised neonates to another hospital. The acute nature of neonatal stroke, where even a few minutes can be critical, makes such a practice extremely problematic.

However, the present invention may be used in combination with a cool cap system, for example, the Olympic Cool-Cap System, which is a helmet designed to provide hypothermia therapy for neonatal encephalopathy caused by hypoxic-ischemic encephalopathy (HIE), preventing cerebral palsy in babies born with little or no oxygen. The instant invention and the cool cap can be used together to provide additive or even synergistic benefits.

The irreversible cell damage that leads to neuronal death are manifold, but can be categorized into two main areas: protonic stress from ATP hydrolysis and lactic acid accumulation, activation of lysosomal proteolytic activity, which causes cell lysis, and free radical damage by radicals, usually, but not necessarily limited to, oxygen radicals. Influx of calcium ions, discussed earlier as the result of ATP losses and failure of ion pumps, triggers proteolytic damage to cells. Radical damage stems from three sources, all related to ATP metabolism and ATP losses.

The basic oxidative phosphorylation process in the mitochondria is not 100% efficient; the result is that some free radicals are continually being released into the cell and are able to cause some radical damage. This damage can be minimized by adequate antioxidant intake in the diet.

A more serious degree of radical damage occurs during reperfusion. In this instance, the therapeutic treatment of the hypoxic conditions causing neonatal stroke are also inadvertently causing cell damage. In the heart, this is known to be a common occurrence, cardiac reperfusion damage. The biochemical mechanism of this is straightforward: supplying oxygen to a hypoxic tissue, especially a highly oxygen dependent tissue, will cause a surge in oxidative metabolism as oxygen is reduced to water in the cytochrome system (2,3). The surge in metabolism results in a greater than normal release of radicals.

The third type of radical-induced cell damage is mechanistically related to the catabolism of ATP that occurs during neonatal stroke (Scheme 1).

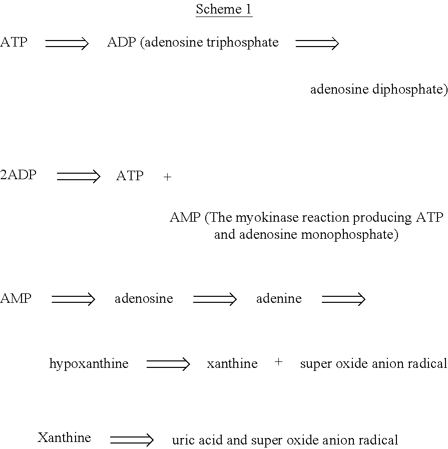

The foregoing biochemical sequence results in two very deleterious events: (1) losses of almost irreplaceable purines from the cell (2) and (2) production of deleterious ROS (super oxide anion radical). The loss of purines from highly energy-depended tissues has been described as a “metabolic disaster (2).” The present invention provides a natural formulation to simultaneously address both of the foregoing issues. The high probability of the same scenario occurring in the brain, with the same type of dire set of circumstances, is addressed herein.

Superoxide dismutase (SOD) is the enzyme that inactivates superoxide radical anion; however, the product, hydrogen peroxide is a reactive oxygen species (ROS), and furthermore can cause the formation of the most reactive of all ROS, the hydroxyl radical (HO⁻), by the Fenton reaction:

Fe²⁺+H₂O₂→Fe³⁺+HO⁻+OH⁻

Therefore, paradoxically, the loss of oxygen supply to the brain, during neonatal stroke, leads to cell damage by oxidation via ROS. This phenomenon, in addition to proteolytic damage and protonic stress by excessive lactic acid accumulation and ATP hydrolysis discussed earlier, is responsible for the irreversible cell damage of neurons, in turn, the causes of the disabilities of children explained earlier.

### Compositions of the Invention

Dietary ribose has been shown to slow losses of ATP during strenuous exercise, and other physiological states where oxygen supplies are limiting and cells would otherwise experience damage. Ribose acts in two ways: stimulation of resynthesis of ATP and by trapping molecules inside the cell that are needed for the resynthesis of ATP (e.g., preventing the loss of uncharged purines from the cell). Maternal dietary ribose will have a dual beneficial effect: both mother and baby will experience boosts in energy levels and decreases in cell damage, especially via ROS and proteolysis.

In one embodiment, modest but effective levels of ribose, along with an optional antioxidant, including a broad spectrum of antioxidants, administered by, for example, diet, will lessen or prevent brain damage in babies if neonatal stroke should occur. Because it is difficult to predict all, or even a majority, of cases of neonatal stoke, dietary supplementation should begin three weeks prior to the expected date of parturition.

Other than the prevention or lessening of the effects of perinatal/neonatal stroke, other benefits will be realized by the compositions and methods of the invention. Maternal health will be improved by this treatment by helping aid in the reduction of ROS and supplying more energy for the smooth muscles involved in the birthing process. Likewise, limitations of the growth rate and prenatal development, differentiation and growth, all ATP requiring processes, will be circumvented by the invention. Furthermore, any potential transfer of ROS from the mother to the fetus will be reduced.

As discussed above, compositions of the invention include ribose and optionally one or more antioxidants.

Ribose

Ribose is an organic compound with formula C₅H₁₀O₅; specifically, a monosaccharide (simple sugar) with linear form H—(C═O)—(CHOH)₄—H, which has all the hydroxyl groups on the same side in the Fischer projection.

The term may refer to any of two enantiomers: preferably to D-ribose, that occurs widely in nature (is synthesized by each and every cell in the body); or to its synthetic mirror image L-ribose, which is not found in nature.

D-ribose was first reported in 1891 by Emil Fischer. It comprises the backbone of RNA, a biopolymer that is the basis of genetic transcription. It is related to deoxyribose, as found in DNA. Once phosphorylated, ribose can become a subunit of ATP, NADH, and several other compounds that are useful in metabolism.

In one embodiment, about 0.1 to about 100 grams of ribose are administered daily starting approximately three weeks prior to the due date of the infant. In one embodiment, ribose is co-administered with one or more antioxidants.

Antioxidants

An antioxidant is a molecule capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols.

Although oxidation reactions are useful for life, they can also be damaging; hence, plants and animals maintain complex systems of multiple types of antioxidants, such as glutathione, vitamin C, and vitamin F as well as enzymes such as catalase, superoxide dismutase and various peroxidases.

Antioxidants include, but are not limited to vitamins (e.g., vitamin A (retinol), beta-carotene, vitamin C (ascorbic acid), vitamin E (including tocotrienol and tocopherol)); vitamin cofactors and minerals (e.g., coenzyme Q10, manganese, iodide); hormones (e.g., melatonin); carotenoid terpenoids (e.g., alpha-carotene, astaxanthin, beta-carotene, canthaxanthin, lutein, lycopene, zeaxanthin); flavonoid polyphenolics (e.g., flavones (e.g., apigenin, luteolin, tangeritin); flavanols (e.g., isorhamnetin, kaempferol, myricetin, proanthocyanidins, quercetin); flavanones (e.g., eriodictyol, hesperetin, naringenin); flavanols and their polymers (e.g., catechin, gallocatechin and their corresponding gallate esters, epicatechin, epigallocatechin, theaflavin, thearubigins); isoflavone phytoestrogens (e.g., daidzein, genistein, glycitein); stilbenoids (e.g., resveratrol, pterostilbene); anthocyanins (e.g., cyanidin, delphinidin; malvidin, pelargonidin, peonidin, petunidin); phenolic acids and their esters (e.g., chicoric acid, chlorogenic acid, cinnamic acid, ferulic acid, ellagic acid, ellagitannins, gallic acid, gallotannins, rosmarinic acid, salicylic acid); nonflavonoid phenolics (e.g., curcumin, flavonolignans (e.g., silymarin), xanthones—mangosteen, eugenol); and other organic antioxidants (e.g., bilirubin, citric acid, oxalic acid, phytic acid, lignan, N-acetylcysteine, R-alpha-lipoic acid, uric acid).

The antioxidants for use in the methods/compositions of this invention are chosen to represent a broad spectrum of properties: solubilities, stereochemical characteristics, and reactivities. These properties were chosen as cellular damage by ROS can occur in the cytoplasm, an aqueous environment, in membranes, a hydrophobic environment, or at interfaces between these two extremes. Indeed some enzymes, including mitochondrial creatine kinase (mitochondrial CK), exhibit an unusual degree of hydrophobicity because they are normally membrane bound. The set of antioxidants in this invention are chosen to be water-soluble, fat-soluble, and those whose solubilities are partitioned between these two extremes to varying degrees.

In one embodiment, the water-soluble antioxidants include, but are not limited to (a) ascorbate (which can be administered at about 0.1 to about 30 grams per day maternally starting approximately three weeks prior to the due date of the infant); and (b) catechins, (+)-chatechin, pyrogallol, (−)-epicatechin, (−)-epigallocatechin, (−)-epicatechin gallate and (−)-epigallocatechin gallate), resveratrol, furanones (2,5 dimethyl-4-hydroxy-3(2H)-furanone and 2-ethyl-4-hydroxy-5-methyl-3(2H)-furanone), hydroxyhydroquinone (which can be administered at about 0.1 to about 5.0 g, for each, starting approximately three weeks prior to the due date of the infant).

In one embodiment, the lipid-soluble antioxidants include, but are not limited to (a) tocols, tocotrienols and tocopherols, where the tocotrienols predominate (which can be administered at about 0.1 to about 10 grams per day total tocols maternally and neonatally, orally, cutaneously); (b) carotenoids, including lycopene, beta-carotene, lutein, zeaxanthin, neoxanthin, etc. (there are several hundred known carotenoids) (which can be administered at about 0.1 to about 10 grams per day total maternally and neonatally, orally, subcutaneously); and (c) BHA, BHT and other synthetic antioxidants (which can be administered at about 10 mg to about 1 g per day or about 0.1 to about 10 grams per day maternally, and neonatally, subcutaneously).

In one embodiment, the partitionable antioxidants include, for example, trolox (6-hydroxy-2,5,7,8-tetramethylchroman-2-carboxlic acid). In another embodiment, the antioxidant includes, for example, natural extracts and cold-pressed oils rich in lipid-soluble natural antioxidants and seed aqueous extracts rich in water-soluble antioxidants; also, partitionable antioxidants from both sources, for example, seeds.

In one embodiment, the composition includes mixtures of two or more of the chemicals, antioxidants or extracts mentioned herein in combination with ribose. In one embodiment, there is synergy between ribose and the one or more antioxidants or extracts. In one embodiment, the there is synergy among the antioxidants and/or extracts. In one embodiment, the compositions of the invention are administered to the expectant mother, to the newborn infant and/or to the unborn baby. The route of administration as discussed below can be any feasible route including orally, cutaneously, intravenously and/or intramuscularly.

Various combinations of antioxidants with various solubility characteristics and partionability characteristics can made to conform to certain required or desired solubilities by processes such as microencapsulation as in “Fat substitutes containing water soluble beta-carotene,” U.S. Pat. No. 5,532,009, which is incorporated herein by reference.

### Formulation and Administration Route

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and/or α-glycerophosphate. Suitable inorganic salts may also be formed, including hydrochloride, hydrobromide, sulfate, nitrate, bicarbonate, and/or carbonate salts.

Pharmaceutically acceptable salts may be obtained using standard procedures well known in the art, for example, by reacting a sufficiently basic compound such as an amine with a suitable acid affording a physiologically acceptable anion. Alkali metals, for example, sodium, potassium or lithium, or alkaline earth metal salts, for example calcium, of carboxylic acids can also be made.

Compounds of the present invention can conveniently be administered in a pharmaceutical composition containing the compound in combination with a suitable excipient/carrier. Pharmaceutical compositions containing a compound appropriate for use herein are prepared by methods and contain excipients/carriers which are available to the art. A generally recognized compendium of such methods and ingredients is Remington's Pharmaceutical Sciences by E. W. Martin (Mark Publ. Co., 15^{th} Ed., 1975). The compounds and compositions of the present invention can be administered parenterally, for example, by intravenous, intraperitoneal or intramuscular injection, topically, orally, or rectally.

For oral therapeutic administration, the active compounds may be combined with one or more excipients/carriers and used in the form of ingestible tablets, buccal tablets, troches, capsules, elixirs, suspensions, syrups, wafers, and the like. Such compositions and preparations typically contain at least about 0.1% of active compound. The percentage of the compositions and preparations may, of course, be varied and may conveniently be between about 2 to about 60% of the weight of a given unit dosage form or about 2 to about 90%. The amount of active compounds in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the invention can be determined by comparing their in vitro activity, and in vivo activity in animal and/or cell models. Methods for the extrapolation of effective dosages in mice, and other animals, to humans are known to the art; for example, see U.S. Pat. No. 4,938,949.

The compounds are conveniently administered in unit dosage form; for example, containing 5 to 1,000 mg, conveniently 10 to 750 mg, including 50 to 500 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compositions can be administered orally or parenterally at dose levels of about 0.1 to 300 mg/kg, including 1.0 to 30 mg/kg of mammal body weight, and can be used in man in a unit dosage form, administered one to four times daily in the amount of 1 to 1,000 mg per unit dose.

For parenteral administration the compounds are presented in aqueous solution in a concentration of from about 0.1 to about 10%, more preferably about 0.1 to about 7%. The solution may contain other ingredients, such as emulsifiers, antioxidants or buffers.

Generally, the concentration of the compound(s) of the invention in a liquid composition, will be from about 0.1-25, or about 0.1-50 or 0.1-80, including from about 0.5-10, weight percent. The concentration in a semi-solid or solid composition such as a gel or a powder will be about 0.1-5 weight percent, including about 0.5-2.5 weight percent.

The exact regimen for administration of the compounds and compositions disclosed herein will necessarily be dependent upon the needs of the individual subject being treated, the type of treatment and, of course, the judgment of the attending practitioner.

### EXAMPLES

The following example is provided in order to demonstrate and further illustrate certain embodiments and aspects of the present invention and are not to be construed as limiting the scope thereof.

Protective Effects of a Nutraceutical, with a Focus on Hypoxia and Ischemia/Reperfusion Injury

### Introduction

The purpose of this study was to perform an in vitro study on a nutraceutical product, to examine its protective effects in situations mimicking or reflecting hypoxia and ischemia/reperfusion injury.

The instant invention aims at use in pregnant mothers to protect the baby during parturition from brain damage, inflammation, and life-threatening complications.

During the birthing process, two scenarios may contribute to such complications. One is hypoxia, defined as reduced oxygen pressure in the cord blood; the other is pinching of the cord to create a transient ischemic condition with intermittent lack of oxygen delivery to the baby. The hypoxia on its own triggers cellular signals that may initiate programmed cell death (apoptosis), whereas in the ischemic situation it is the reperfusion that triggers excessive free radical damage and inflammation from endothelial and inflammatory cells, primarily neutrophil granulocytes.

The nutraceutical product tested (e.g., ribose) contains compounds that may support cellular energy production and protects from apoptosis. The test product also contains antioxidant vitamins that may have additional protective effects against stroke and vascular endothelial effects.

### Materials and Methods

Ribose was obtained from Heartland, Minneapolis, Minn. Tocotrienols were purchased from Vitamin Research Products Inc., which sells Anatto tocotrienols (predominantly delta-tocotrienol).

Product handling—Initial attempts involved using published methods on how best to introduce the highly lipophilic tocotrienols into cell culture medium to allow in vitro cell-based work. Tocotrienols were suspended in 95% ethanol, and then mixed with bovine serum albumin for extended time periods. Subsequently, ethanol concentration must be reduced to 2% or less in cell cultures, to avoid negative effect by ethanol. This was done using physiological saline.

Next, the Tocotrienol/Ethanol blend was mixed into cell culture medium containing 10% serum. This worked very well and was used for all subsequent experiments. This method allowed the obtainment of data showing: antioxidant content, antioxidant protection, reduced free radical formation, increased cell viability under oxidative stress, and using cell lines also show evidence for certain dose ranges that had protective effects against hypoxic conditions.

### Results/Discussion

1a. Chemical Antioxidant Capacity

The rationale behind the method is similar to the ORAC (Oxygen Radical Absorbance Capacity) test for peroxyl free radicals. A cell-free version of the CAP-e (Cell-based Antioxidant Protection in Erythrocytes) assay was used. It allows assessment of antioxidant potential in a method that is comparable to the ORAC test, but uses the exact same product preparation, dye, and reaction times as the cell-based CAP-e assay (see 1b, below). Comparing data from the two methods shows whether a product contains antioxidants and if yes, then whether these can enter into live cells and protect these from oxidative stress.

The test products were prepared in serial 5-fold dilutions. The DCF-DA dye, which turns fluorescent upon exposure to reactive oxygen species, was added. Oxidation was triggered by addition of the peroxyl free radical generator AAPH (2,2′-azo-bis(2-amidino-propane)dihydrochloride). The fluorescence intensity was evaluated. The low fluorescence intensity of untreated control wells served as a baseline, and wells treated with AAPH alone served as a positive control for maximum oxidative damage, if a reduced fluorescence intensity of wells exposed to a test product and subsequently exposed to AAPH is observed, this indicates that the test product contained antioxidants able to interfere with peroxyl free radicals.

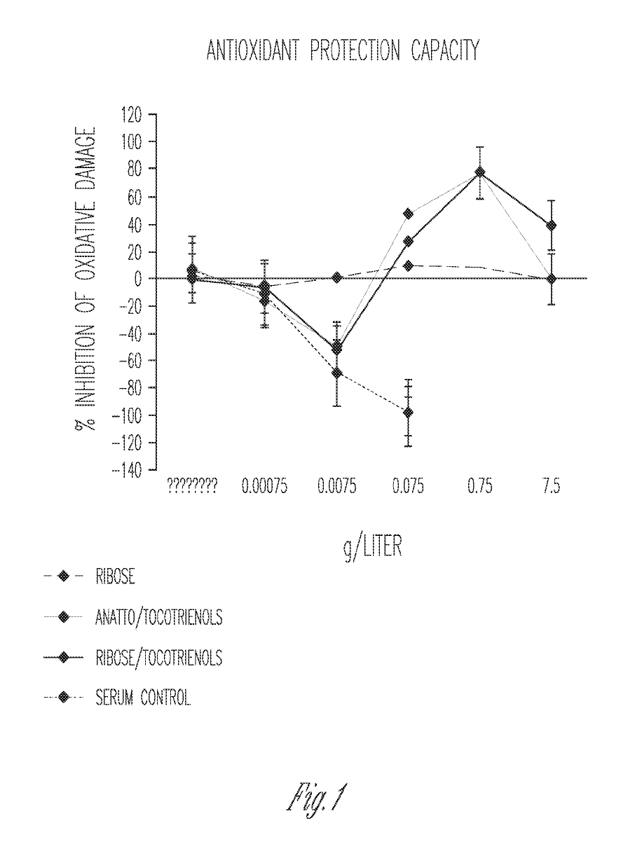

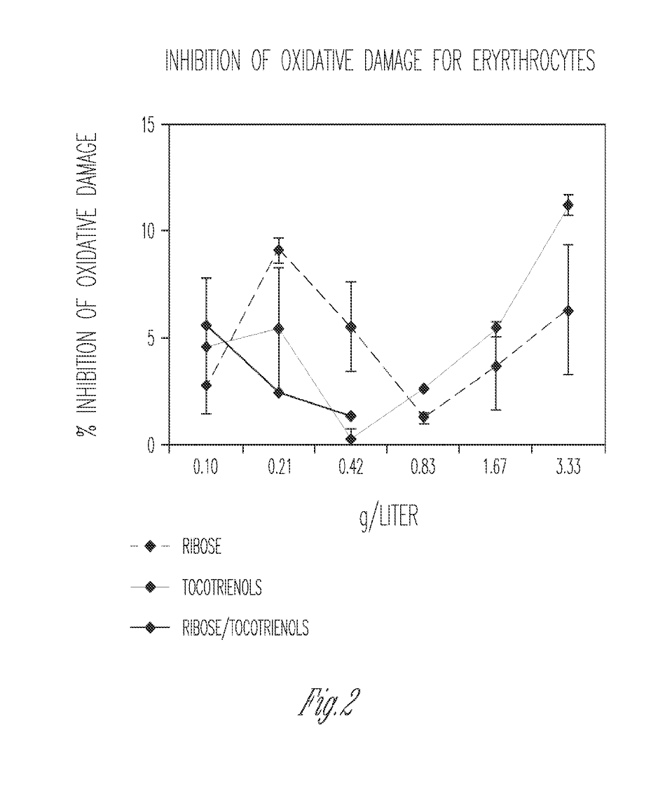

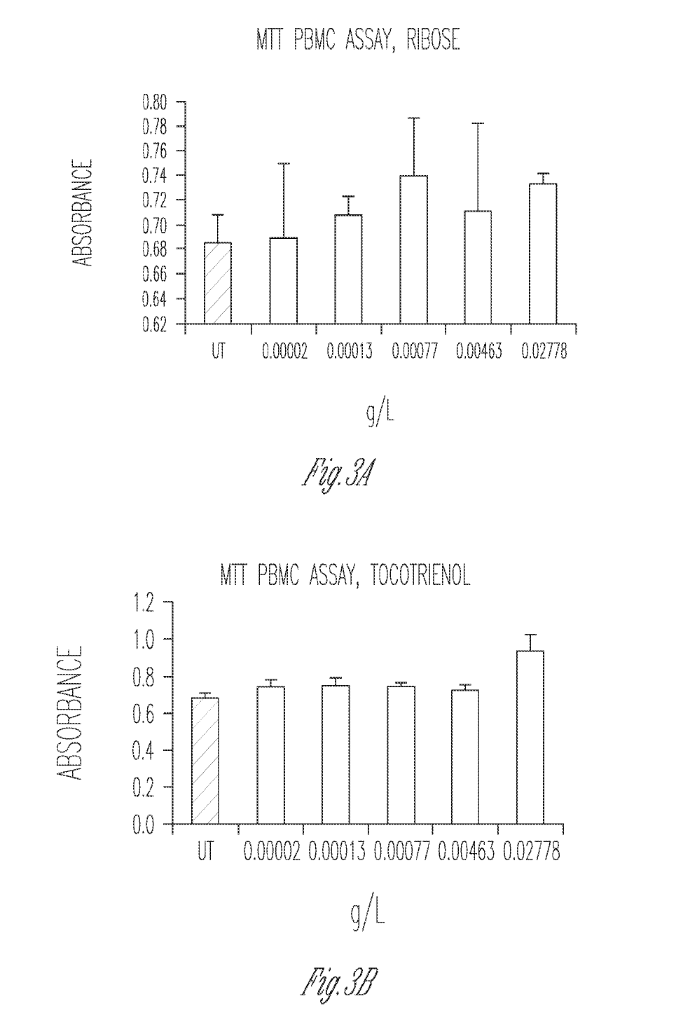

FIG. 1 depicts a graph which shows the total antioxidant protection capacity against peroxyl free radicals for each ingredient, the blend, and a serum control. It can be seen that the serum interfered with the assay; despite this interference, the tocotrienols and the blend were able to reduce the oxidative damage in the assay by over 80%.

1b. Cell-Based Antioxidant Protection Assay CAP-c

The rationale behind the method is that it allows assessment of anti-oxidant potential in a method that is comparable to the ORAC test, but only allows measurement of anti-oxidants that are able to cross the lipid bilayer cell membrane. As a model cell type, red blood cell (RBC) was used. This is an inert cell type, in contrast to other cell types such as PMN cells as described in section 2, where pro-inflammatory compounds may induce the reactive oxidative burst, or anti-inflammatory compounds may perform cellular signaling and change the behavior of the PMN cell, at doses many times below levels of detection for antioxidants. This assay was developed particularly to be able to assess antioxidants from complex natural products in a cell-based system.

Human RBC were washed repeatedly physiological saline, and then exposed to the test products. During the incubation with the test products, any antioxidant compounds able to cross the cell membrane will enter the interior of the RBC. Then the RBC are washed to remove compounds that were not absorbed by the cells, loaded with the DCF-DA dye, which turns fluorescent upon exposure to reactive oxygen species. Oxidation was triggered by addition of the peroxyl free radical generator AAPH. The fluorescence intensity was evaluated. The low fluorescence intensity of untreated control cells served as a baseline, and RBC treated with AAPH alone served as a positive control for maximum oxidative damage. If a reduced fluorescence intensity of RBC exposed to a test product and subsequently exposed to AAPH was observed, this indicates that the test product contained antioxidants available to penetrate into the cells and protect these from oxidative damage.

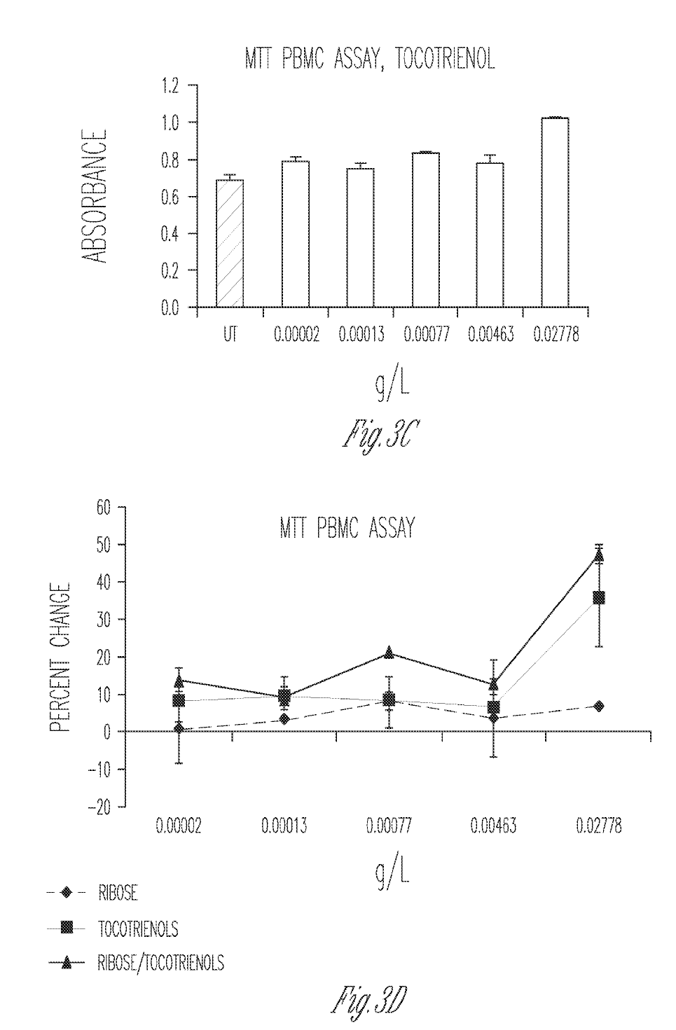

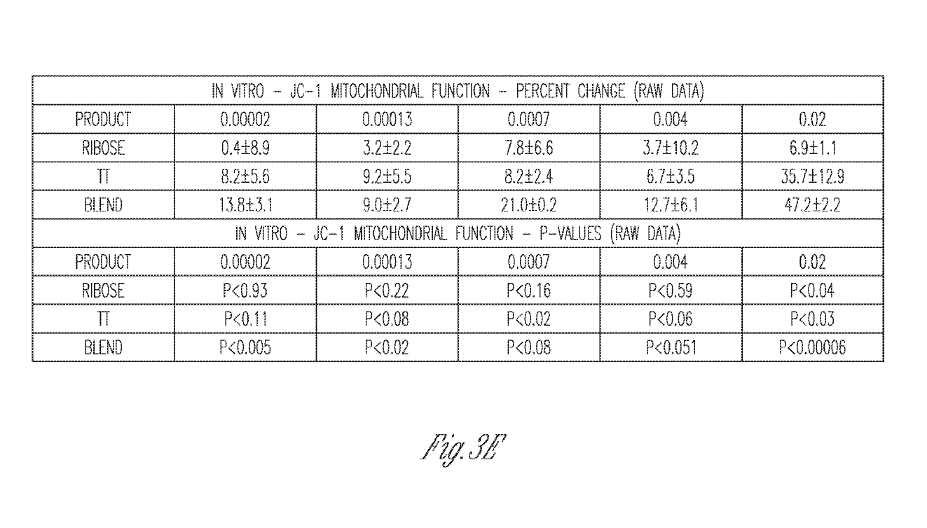

A biphasic response of antioxidant protection was seen in the cell-based system, where higher and lower doses of each ingredient provided protection. The blend provided protection at the lowest doses tested, suggesting that the blend may be effective in protecting live cells from oxidative damage at even lower doses than tested here. FIG. 2 provides a dose response graph.

Thus, both ribose and tocotrienol protected the erythrocytes. This illustrates the protective properties of both compounds against radical damage by ROS and, therefore, for other cells, most especially the white blood cells, the reduction of inflammatory reactions. Furthermore, because the erythrocyte is the sole oxygen delivery cell for all cells, including nerve cells, the health, viability, and oxidation state (ferrous vs. ferric) of the erythrocyte is important. Ribose, tocotrienol, and the blend, all imparted protection and therefore will aid in the delivery of oxygen to cells.

This relates to the previous discussion on NO as NO is vasodilatory. Dilation, if optimum, aids in relieving ischemia but needs healthy, energetic red cells to carry oxygen to the tissue.

1c. Evaluation of Protection of Cellular Viability in the Presence of Oxidative Stress.

Oxidative damage can trigger premature cellular death by a mechanism called apoptosis (programmed cell death). This death pathway can be monitored by highly specific cellular markers. Protection from cell death can be monitored as delay or absence of these markers.

Apoptosis is a carefully regulated process of cell death that occurs as a normal part of cellular development. In contrast to necrosis, a form of cell death resulting from acute cellular injury, apoptosis is carried out in an ordered process that is generally advantageous during an organism's life cycle. An example of apoptosis in an organism is the loss of webbing between fingers in a human.

The human vascular anticoagulant, annexin V, is a Ca²⁺-dependent phospholipid-binding protein that has a high affinity for phosphatidylserine. In normal viable cells, phosphatidylserine is located on the cytoplasmic surface of the cell membrane. However, in apoptotic cells, PS is translocated from the inner to the outer leaflet of the plasma membrane, thus exposing PS to the external cellular environment. Annexin V labeled with a fluorophore can identify apoptotic cells by binding to phosphatidylserine exposed on the outer leaflet. AnnexinV-FITC was used to label apoptotic cells. Co-staining with PI or 7AAD, which only stains cells at a late phase of cell death, allows one to distinguish early and late apoptosis. Cells staining only with PI or 7AAD, without Annexin V, are necrotic cells.

Among freshly isolated human PBMC and PMN cells, a proportion was already on an apoptotic path. When cultured in vitro, these cells will continue the apoptotic process. H₂O₂ was added to trigger oxidative stress-induced apoptosis, and assess whether the test product was able to protect the viability of cells that are under severe oxidative stress. The testing was performed where each testing condition, including each serial dilution of test product, was performed in triplicate. The experiment was performed once on cells from a healthy donor.

Since multiple repeats were needed, as well as hypoxia work on the A172 microglial cell line (see data below), the method for assessment of cell viability was changed to the cheaper and faster MTT assay.

The MTT assay is a colorimetric assay for measuring the activity of enzymes that reduce MTT or similar dyes (XTT, MTS, WSTs) to formazan dyes, giving a purple color. A main application allows assessment of the viability and the proliferation of cells in culture. The assay can also be used to screen for cytotoxicity of potential medicinal agents and toxic materials, since those agents would stimulate or inhibit cell viability and growth.

The data presented in FIGS. 3a-e show that both ingredients protect cellular viability and metabolic function under conditions of oxidative stress. The data also show that the blend of the two ingredients performed better than each ingredient alone.

2a. Reactive Oxygen Species (ROS) Formation in Polymorphonuclear (PMN) Cells

PMN cells are complex and capable of reacting in several ways upon exposure to natural products as follows: 1. Passive absorption of antioxidants into the cells, neutralizing ROS within the cells; 2. Active signaling leading to increased ROS production; 3. Active anti-inflammatory signaling leading to a reduced production of ROS.

Many natural products with antioxidant capacity also reduce the ROS formation in inflammatory cells. However, other products may actually increase the ROS formation, despite antioxidant capacity, and this may indicate and interesting cooperation between support of antimicrobial defense mechanisms and antioxidant capacity.

This is in contrast to the CAP-e assay (shown above), where only the antioxidants able to penetrate the cells are measured, without the superimposition of cellular signaling. Thus, a logical sequence of testing is to first perform the CAP-e assay, and then perform the more complex ROS PMN assay.

Freshly purified human PMN were exposed to the test products. During the incubation with a test product, any antioxidant compounds able to cross the cell membrane can enter the interior of the PMN cells. Any compound that mediates a signal by engaging cell membrane receptors on the outside of the cell can do so.

Then the cells were washed to remove unbound and unabsorbed test compounds, loaded with the DCF-DA dye, which turns fluorescent upon exposure to reactive oxygen species. Oxidation was triggered by addition of H₂O₂. The fluorescence intensity of the PMN cells was evaluated by flow cytometry. The low fluorescence intensity of untreated control cells served as a baseline and PMN cells treated with H₂O₂ alone served as a positive control. If the fluorescence intensity of PMN cells exposed to an extract, and subsequently exposed H₂O₂, was reduced compared to H₂O₂ alone, this indicates that a test product has anti-inflammatory effects. In contrast, if the fluorescence intensity of PMN cells exposed to a test product was increased compared to H₂O₂ alone, this indicates that a test product has pro-inflammatory effects.

The testing was performed using a broad range of serial dilutions of products. Testing was completed once on cells from a healthy donor.

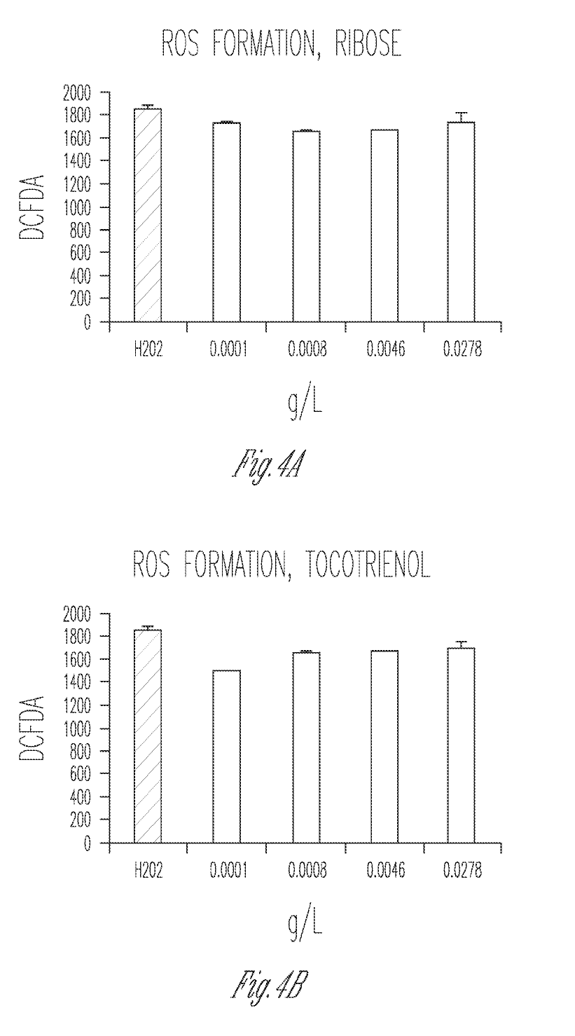

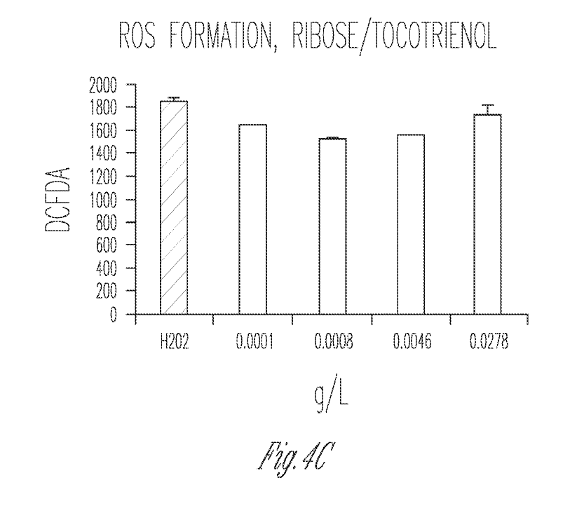

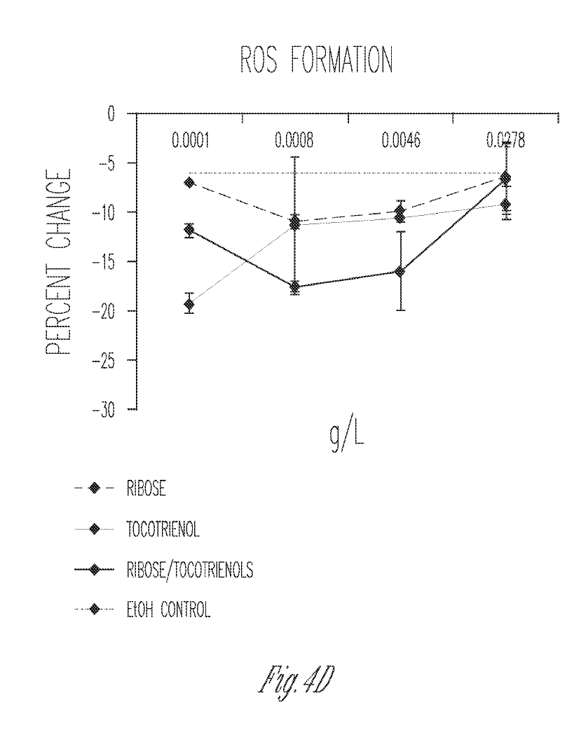

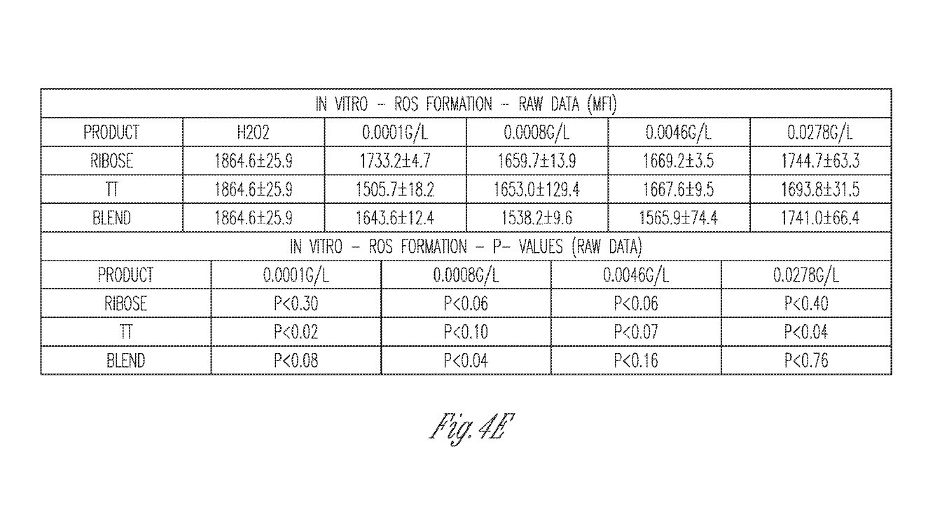

The results are presented in FIGS. 4a-e. All products tested were shown to reduce ROS formation under oxidative stress. When products were incubated with PMN cells for a period of 20 minutes it was determined that the blend performed similar to ribose but with a greater effect at reducing ROS due to the addition of TT. However, TT was found to reduce (19%) ROS formation the most, at the least concentrated dilution (0.0001 g/L) and the data was found to be statistically significant (p<0.02).

### BIBLIOGRAPHY

(1) Leninger, A. L. 1982. Principles of Biochemistry, Worth Publishers, Inc. New York.
(2) Ingwall, J. A. 2002, ATP and the Heart. Kluwer Academic Publishers. Norwell, Mass.
(3) Addis, P. B. 2007, The Health Benefits of Ribose. Basic Health Publications, Laguna Beach, Calif.
(4) Hall, N. P., et al. 1977. Biochemical and Biophysical Research Communications, 76(3):950-956.
(5) Hall, N. P. et al. 1979. Biochemistry, 18:1745-1751.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method comprising administering to a subject in need thereof an effective amount of ribose to reduce risk of neonatal stroke or reduced risk of sequellae of neonatal stroke, wherein the subject in need thereof is an expecting mother.

2. The method of claim 1, wherein the sequellae of neonatal stroke is death, cerebral palsy, learning disabilities, mental retardation, seizure disorders, schizophrenia, hearing (e.g., deafness) and visual disorders, attention-deficit disorders (including, for example, hyperactivity), epilepsy or a combination thereof.

3. A method comprising administering to an expectant mother an amount of ribose to improve the growth, vigor, intelligence, overall health, resistance to disease in the neonate.

4. A method comprising administering to a subject in need thereof an effective amount of ribose to reduce oxidative stress by reduction of ROS, wherein the subject in need thereof is an expecting mother or a neonate.

5. The method of claim 4, wherein the reduction of oxidative stress yields a reduction in tissue and DNA damage and/or a reduction in risk of cancer.

6. The method of any one of claims 1-5 wherein the ribose provides protection and/or stabilization of cell membranes of neurons and microglia, protection and/or stabilization of membrane-bound mitochondrial creatine kinase (CK) of neurons and microglia and/or maintenance of fluidity of cell membranes.

7. The method of any one of claims 1-5, further comprising the administration of one or more antioxidants.
